(19) United States
(12) Patent Application Publication
(10) Pub. No.: US 2023/0410244 A1
(43) Pub. Date: Dec. 21, 2023

(54) MEDICAL IMAGING WITH DISTRIBUTED CLUSTER-BASED PROCESSING

(71) Applicant: Siemens Healthcare GmbH, Erlangen (DE)

(72) Inventors: Swen Campagna, Engelthal (DE); Armin Stranjak, Uttenreuth (DE); Yan Tu Huang, Shenzhen (CN)

(21) Appl. No.: 18/199,951

(22) Filed: May 20, 2023

(30) Foreign Application Priority Data
May 20, 2022 (EP) 22174571.4

(51) Int. Cl. G06T 1/20; G16H 30/40; G06F 9/48; G06T 11/00

(52) U.S. Cl. CPC G06T 1/20; G16H 30/40; G06T 2210/52; G06T 11/003; G06T 2210/41; G06F 9/4887

(57) ABSTRACT

A local control device for a medical imaging system includes a measurement control unit for dividing a computing task into a sequence of consecutive sub-tasks. The computing task is related to a generation of image data based on measurement data. The local control device includes an execution unit for executing the computing task. The local control device includes a selection unit for selecting a sub-task of the consecutive sub-tasks for external execution. The local control device includes an outsourcing unit for outsourcing the selected sub-task to a remote computing source for remotely generating a partial computing result and for receiving the generated partial computing result. The execution unit is arranged to execute the computing task based on the received generated partial computing result.

This application claims the benefit of European Patent Application No. EP 22174571.4, filed on May 20, 2022, which is hereby incorporated by reference in its entirety.

### BACKGROUND

The present embodiments relate to a local control device for a medical imaging system. The present embodiments also concern a medical imaging system. Further, the present embodiments relate to a system-cluster.

Further, the present embodiments relate to a method for controlling a medical imaging system.

Medical imaging processes (e.g., MR imaging processes) depict the inside of the body of a patient, either in full or in part. Imaging techniques help doctors to diagnose a disease, to assess its severity, and to monitor sick patients. Most imaging procedures are painless, relatively safe, and non-invasive. Medical imaging includes, for example, imaging techniques based on radiation, ultrasound scans, and magnetic resonance imaging (abbreviated as MM or MR imaging).

Imaging modalities (e.g., magnetic resonance imaging systems (abbreviated by MRI system or MR system)) are to have substantial computing power for quickly performing preparation, execution, and evaluation (e.g., a reconstruction of images based on raw data) of an imaging process. It is important that reconstructed images are available to the operator of a scan unit shortly after executing the measurement (e.g., the acquisition of raw data). Short time of an image reconstruction is crucial (e.g., in emergency circumstances where patient's health could be at risk).

For a magnetic resonance imaging system, two different approaches are conventionally used for achieving a sufficient speed of reconstruction. The first is to equip an MR system with sufficient local centralized computing power, basically related to the number of receive channels. Receive channels are used for receiving magnet resonance signals from an examination portion. However, that approach needs many resources.

The second approach includes an offloading of the computing task, especially the image reconstruction, somewhere else (e.g., somewhere in the cloud, such as in a cloud computing system). Hence, the MR system itself requires less computing power, but it is dependent on an external computing resource, which is to be paid also. Further, either bandwidth limitations or poor network structure impede this approach significantly.

Conventionally, a computing task (e.g., an image reconstruction) is designed in a monolithic computing model as a default (e.g., the whole computation is run as a single complete computing task).

In FIG. 1, an MR system with an associated computer, also referred to as measurement and reconstruction system (abbreviated by the acronym MARS), is depicted. Such measurement and reconstruction system may include two separate computers or one single computer. In both cases, the reconstruction is typically implemented monolithically (e.g., executed on one single computer).

It is desirable to have enough capacities for executing the necessary calculations for imaging independently; however, it is desirable to be capable also to use external resources for accelerating the imaging process. Today, a computer of a medical imaging system may be able to communicate with external resources such as a cloud computing system or other medical imaging systems. However, an external calculation of an important process makes the medical imaging system dependent on a present capacity of communication lines and on the capacity of the remote resources. However, reliability of schedule of a control and an imaging generating process is crucial for an efficient use of medical imaging capacities.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an increase of speed of medical imaging while providing the reliability of imaging operations are provided.

A local control device for a medical imaging system includes a measurement control unit for dividing a computing task into a sequence of consecutive sub-tasks. As later described, the sub-tasks may be independent of each other (e.g., the sub-tasks may include independent images). Thus, the ordering into a sequence is arbitrary in that case. As a local control device, a control device that is positioned at the location of an assigned medical imaging system and is intended to control the assigned medical imaging system is provided. The computing task is related to the generation of image data based on measurement data and, for example, to a control task for controlling an imaging process or a task for processing measurement data for generating image data. For example, the computing task is related to the generation of control data to be transmitted to a scan unit and/or to the processing of raw data received from the scan unit. As later discussed in detail, the computing task related to the process of generating image data may include a reconstruction task or a filtering task.

For example, a complicated reconstruction task (e.g., a parallel reconstruction) may divided into sub-tasks.

A parallel reconstruction may be applied to an acquisition of different images in parallel (e.g., by interleaving k-space lines of different images). A parallel reconstruction may also be applied to a computationally parallel image reconstruction of different images for whatever reason (e.g., because of a 3D-Fast Fourier Transformation or because raw-data flows in so rapidly), while the reconstruction of a single image is very computationally intensive, and thus, different images may be reconstructed in parallel then.

A complicated reconstruction task may also include an MRI “parallel imaging” that is an acquisition technique to generate an image by reducing the number of acquired k-space lines (e.g., by the use of multiple receive channels). Image reconstruction for this task is quite compute intensive; therefore, image reconstruction is also advantageously divided in sub-tasks and distributed to different computing sources.

The sequence of consecutive sub-tasks is referred to as the order in which the sub-tasks are processed, if the sub-tasks are all sequentially performed, only by the local control device. However, it is intended to distribute at least one sub-task to a remote computing source such that some sub-tasks are possibly processed in parallel.

Optionally, the local control device may include a first communication interface for transmitting the control data to a scan unit of the medical imaging system and for receiving the raw data from the scan unit.

Further, the local control device includes an execution unit for executing the computing task. The execution of the computing task includes a generation of computing results related to the generation of image data (e.g., related to the generation of control data and/or to the processing of received raw data).

The local control device also includes a selection unit for selecting a sub-task of the consecutive sub-tasks for external execution. As later described, such a sub-task may be selected based on the order of the reception of input data necessary for execution of the selected sub-tasks or based on the time consumption of the selected sub-tasks. In one embodiment, a sub-task with high time consumption is selected for external execution and/or a sub-task that is positioned on the back positions of a sequence of sub-tasks is selected for external execution.

Further, the local control device includes an outsourcing unit for outsourcing the selected sub-task to a remote computing source for remotely generating a partial computing result and for receiving the generated partial computing result. A remote computing source may be any computing source outside of the medical imaging system that includes the local control device. For outsourcing the sub-task, the outsourcing unit may include a second communication interface for communication with a remote computing source.

The selection unit and/or the outsourcing unit may be encompassed by the measurement control unit, which may also execute the scheduling tasks of selecting and/or outsourcing the sub-tasks.

The remote computing source may include a set of variable computing resources that is not fixed, but may and should adapt dynamically to the availability and thus scales dynamically and may even be extended to further computing resources if required (e.g., on pay-per-use-basis in a cloud computing system). In one embodiment, the local control device for a medical imaging system itself offers its computing capabilities to other medical imaging systems if it is idle itself.

Hence, the local control device is arranged to execute at least one computing task that is divided into a sequence of consecutive sub-tasks, and to transmit at least one sub-task to at least one remote computing source, for example, through the second communication interface.

The local control device is arranged to receive computing results carried out by occasion in the at least one remote computing source (e.g., through the second communication interface) and to use the received computing results for execution the computing task based on the received generated partial computing result. However, if the computing results of the remote computing source are not available due to any problems of data transmission or data procession or due to any other obstacle for an availability of the remotely computed results, the computing task is completed by the local control device as substitute. In other words, the local control device performs the complete computing task by default.

In one embodiment, the local control device is always self-contained and always able to execute the overall computing task on its own. The local control device uses available free computing sources dynamically that are anyhow available or are intentionally provided to speed-up computations. Further, computing resources of medical imaging systems that are usually only used when an imaging process is running may be exploited over the whole time if the computing resources are accessed by remote medical imaging systems. In one embodiment, time critical tasks in medical imaging (e.g., the execution of an image reconstruction) are accelerated using the remote computing source if available such that the results (e.g., images) are available to an operator of the medical imaging system shortly after executing the measurement itself. Short time of execution of computing tasks (e.g., tasks for executing a reconstruction) is to be provided, especially in emergency circumstances where patient's health is at risk.

The medical imaging system (e.g., an MR imaging system) according to the present embodiments includes a scan unit for acquiring raw data from an object to be examined and the local control device according to the present embodiments. If the medical imaging system includes an MR imaging system, the local control device may include a measurement control unit and an execution unit (e.g., a reconstruction unit) for reconstructing image data based on acquired raw data. The medical imaging system shares the advantages of the local control device according to the present embodiments.

The medical imaging systems may also include more than one medical imaging system (e.g., an ensemble of at least two medical imaging systems). In that case, at least one of the at least two medical imaging systems includes a local control device also including an execution unit used as the remote computing source for remotely generating a partial computing result of a selected sub-task received from another medical imaging system. Also, more than one (e.g., all) of the at least two medical imaging systems may use their local control device and, for example, their execution unit as a remote computing source for remotely generating a partial computing result of a selected sub-task of another remotely located medical imaging system.

In one embodiment, the medical imaging system includes an ensemble of at least two medical imaging systems. Each of the at least two medical imaging systems includes the local control device according to the present embodiments and an execution unit also used as a remote computing source for remotely generating a partial computing result of the selected sub-task of another medical imaging system. In one embodiment, the different medical imaging systems are enabled to exchange selected sub-tasks dependent on current free calculating capacities such that a medical imaging process of all these medical imaging systems of the ensemble is accelerated as much as possible. In case of a network failure, the system falls back to processing a complete task by one self-contained medical imaging system.

The system-cluster according to the present embodiments includes at least one medical imaging system according to the present embodiments and at least one additional remote system including the remote computing source for remotely generating a partial computing result of the selected sub-task. The remote system includes technical means for performing tasks or sub-tasks outside of the at least one medical imaging system.

In one embodiment, the additional remote system includes a medical imaging system and/or a compute-only source.

As described above, a system-cluster including an ensemble of more than one medical imaging system enables to exchange selected sub-tasks dependent on current free calculating capacities such that a medical imaging process of all these medical imaging systems of the ensemble is accelerated as much as possible. In case of a network failure, the system-cluster falls back to processing a complete task by one self-contained medical imaging system.

A compute-only source does not include a complete medical imaging system. The compute-only source includes only a computer for calculating results of tasks or sub-tasks received from a medical imaging system or another compute-only source. In one embodiment, the at least one compute-only source enables to further speed up the medical imaging process. For example, the system-cluster may include a dedicated computing cluster or one or more highly-performant computing machines with a single task of offering free computing resources that may be used by a medical imaging system that is still self-contained and may operate the complete task on its own in case of network issues. A big advantage of the system-cluster is that the additional computing power (e.g., the additional compute-only source) may easily be extended or renewed with a significantly higher frequency than the medical imaging system itself to benefit from the very short innovation cycles in the computing industry. Such a compute-only source may also be provided by a cloud computing system with higher latencies, but for some use cases, this may also be beneficial, for example, if a very high amount of computation work is required only in rare cases.

The method for controlling a medical imaging system includes the acts of dividing a computing task into a sequence of consecutive sub-tasks, where the computing task is related to a generation of image data based on measurement data. Further, the method according to the present embodiments includes the acts of executing the computing task. For executing the task, a sub-task of the consecutive sub-tasks is selected for external execution. Then, the selected sub-task is outsourced to a remote computing source for remotely generating a partial computing result. The generated partial computing result is received, and the computing task is executed based on the received generated partial computing result (e.g., if there are further computing tasks depending on that result, the act of executing the computing task includes the act of executing the computing task based on the received generated partial computing result). The method for controlling a medical imaging system shares the advantages of the local control device according to the present embodiments.

Some units or modules of the local control device mentioned above may be completely or partially realized as software modules running on a processor of a respective computing system (e.g., on a local control device for a medical imaging system; a magnetic resonance imaging system or a CT-system). A realization largely in the form of software modules may have the advantage that applications already installed on an existing computing system may be updated, with relatively little effort, to install and run these units of the present application. The object of the present embodiments is also achieved by a computer program product with a computer program that is directly loadable into the memory of a computing system, and includes program units to perform the acts of a method of the present embodiments. The acts may include at least those acts that may be executed by a computer (e.g., the acts of dividing a computing task into a sequence of consecutive sub-tasks, executing the computing task, selecting a sub-task of the consecutive sub-tasks for external execution, outsourcing the selected sub-task to a remote computing source for remotely generating a partial computing result, receiving the generated partial computing result, and executing the computing task based on the received generated partial computing result, when the program is executed by the computing system. In addition to the computer program, such a computer program product may also include further parts such as documentation and/or additional components, also hardware components such as a hardware key (e.g., dongle, etc.) to facilitate access to the software.

A computer readable medium such as a memory stick, a hard-disk, or other transportable or permanently-installed carrier may serve to transport and/or to store the executable parts of the computer program product so that these may be read from a processor unit of a computing system. A processor unit may include one or more microprocessors or their equivalents.

The description of one category may also be further developed analogously to the description of another category. In addition, within the scope of the present embodiments, the various features of different exemplary embodiments may also be combined to form new exemplary embodiments.

In a variant of the local control device according to the present embodiments, the control task includes at least one of the following types of tasks: controlling a scan unit of the medical imaging system; and reconstructing image data based on raw data acquired by a scan unit of the medical imaging system.

In one embodiment, the generation of control data or a sub-task of a reconstruction task may be relocated to different computing resources for accelerating a control process or reconstruction process.

As later described in detail, additional acts of a medical image generating process (e.g., a filtering step or a post-processing of generated image data) may be carried out distributed (e.g., in parts by the remote computing source or alternatively, such as by default, by the local control device).

In a variant of the local control device according to the present embodiments, the computing task is executed based on a set of input data, and the measurement control unit is arranged to divide the set of input data into sub-sets and to assign each of the sub-sets of input data to a different sub-task. The input data includes data to be processed by the computing task (e.g., control data or raw data or image data). The separated sub-sets include the data to be processed by the related sub-tasks. In one embodiment, by assigning the related input data to each of the sub-tasks, each of the sub-tasks may be processed independently from each other.

In a variant of the local control device according to the present embodiments, the task includes a reconstruction task for parallel imaging based on sub-sets of parallel acquired raw data, and each of the sub-tasks includes a reconstruction of a partial image based on a different sub-set.

In one embodiment, the execution unit is arranged to locally execute a sub-task if an outsourcing of this sub-task is not appropriate according to a predefined criterion. A predefined criterion may include that the local execution is more efficient (e.g., with respect to aspects of time consumption or existence of free computing resources).

In other words, the outsourced sub-task is locally executed by the execution unit of the local control device by default. “By default” provides that the outsourced sub-task is executed by the local execution unit based on the above-mentioned criterion. The above-mentioned criterion may include the decision of a locally execution of the sub-task in case no result is received from the remote computing unit processing the outsourced sub-task or no result is received under a predefined condition. In one embodiment, the local control unit is self-contained and does not necessarily need the support of any remote computing unit.

In a variant of the local control device according to the present embodiments, the execution unit is arranged to detect, as a special type of a predefined criterion or predefined condition, the current status or expected status of one or more consecutive sub-tasks (e.g., of one or more completed consecutive sub-tasks or one or more consecutive sub-tasks expected to be completed). The consecutive sub-tasks may be sub-tasks preceding the outsourced sub-tasks. The status of one or more of these consecutive sub-tasks is detected to determine based on the detected status if the partial computing result generated by the remote computing source is received by the local control device on time or will be received on time. Hence, the correct timing of the reception of the partial computing result depends on the status of completion of the consecutive sub-tasks (e.g., the sub-task or sub-tasks preceding the outsourced sub-task).

Further, the criterion may also include the condition that the result of the outsourced sub-task is needed for the possibly existing sub-tasks being subsequent to the outsourced sub-task. In one embodiment, at the time point when the execution of the sub-tasks preceding the outsourced sub-task is finished or alternatively at the time point when the execution of the sub-tasks being subsequent to the outsourced sub-task is planned to be started, the result of the outsourced sub-task is to be received according to the predefined criterion.

Hence, it is detected if it is necessary to start the related sub-task by the local control device by default (e.g., as a substitute for the remotely executed sub-task). Further, the local control device is arranged to execute the computing task based on the received partial computing result if the partial computing result is received on time, or alternatively, to execute the selected sub-task itself if the partial computing result is not received on time. In one embodiment, the computing task may be completed in any case without any delay compared to a completely local execution of the computing task.

In another variant of the local control device according to the present embodiments, the sequence of sub-tasks to be processed is implemented as a queue of consecutive sub-tasks. The queue determines an unambiguous order of processing the consecutive sub-tasks (e.g., in case the sub-tasks are performed by the local control device). In one embodiment, an order for processing the different sub-tasks is generated. For example, the order may be determined based on available input data or based on the consumption of time the different sub-tasks need.

In a variant of the local control device according to the present embodiments, the local control device includes a modularized execution unit (e.g., a modularized reconstruction unit).

The modularized execution unit includes a plurality of sub-units, including computing sources for performing a sub-task (e.g., reconstruction sub-tasks). For example, the total imaging generation process includes inter alia a reconstruction task, where, for example, a complete database of input data for that special reconstruction task is already available. As previously discussed, such a reconstruction task may include the parallel reconstruction of a predetermined number of sub-images based on raw data acquired in parallel. This raw data represents the complete database for the different sub-tasks of the reconstruction task.

Some of the different sub-units perform different computing sub-tasks. Hence, a computing task is split into a number of independent computing sub-tasks, where some of the different independent computing sub-tasks are performed by a different computing source (e.g., a different sub-unit). After computation, the results of the different independent computing sub-tasks are combined altogether to a combined final overall single result.

The sub-units may be implemented by a modularized hardware and/or software structure for executing the different sub-tasks. In one embodiment, the execution unit includes individual computers. However, the sub-units may also be implemented by a single computer, where each function of the sub-units is executed by the single computer, but each function is still logically separated. Hence, the execution unit may include a single computer that includes a modularized software structure for individually executing the different sub-tasks.

Hence, the principle of the modularized execution unit is to split up a monolithic task (e.g., a reconstruction task) into certain chunks of independent computing sub-tasks that may be flexibly either executed on a local machine (e.g., the execution unit) or distributed to other places (e.g., remote computing sources) in particular remote medical imaging systems. The generated sub-tasks are already supplied with a necessary data basis of input data such that the generated sub-tasks may be transferred together with the assigned data basis of input data to a logically separated or physically separated computing source.

As mentioned above, the software structure used for the implementation of the distributed structure may include a queue, where the computing task is divided into the above-mentioned sub-tasks and these sub-tasks are stacked up and then distributed to different computing sources. The queue only includes these selected sub-tasks ready to be processed at the point of time of selection. In one embodiment, the individual results of the selected sub-tasks are collected and put together to the final overall result in a reverse manner. The division of the computing task into sub-tasks may be organized by the measurement control unit.

The overall result may include either a simple collection of individual results (e.g., images) for a single measurement or something that is to be at least partly computationally combined or further processed. In one embodiment, the modularized structure of the possibly spatially distributed hardware for executing the computing task is mapped on the software and/or hardware structure of the execution unit of the local control device according to the present embodiments, such that a computing task is enabled to be processed in a modularized manner in each case (e.g., locally for default or by outsourcing at least one sub-task to a remote computing source).

In a further variant of the local control device according to the present embodiments, the remote computing source includes a remote local control unit of a different medical imaging system. In one embodiment, computing resources of medical imaging systems that are usually only used when an imaging process is running may be exploited over the whole time, if the computing resources are accessed by remote medical imaging systems.

In one embodiment, in a variant of the local control device according to the present embodiments, the remote computing source includes a cluster comprising a plurality of local control units for different medical imaging systems. In one embodiment, every medical imaging system that has an idle computing power may be used to execute sub-tasks, and thus, the medical imaging systems form a kind of virtual cluster, distributed and dynamically available. Further, the local control device according to the present embodiments itself may be arranged to offer its computing power to other remote medical imaging systems if it is idle itself.

In a variant of the local control device according to the present embodiments, the remote computing source is located in a cloud computing system. In one embodiment, the remote computing source may also be provided as a virtual resource in a cloud computing system without an actual existence of an additional remote medical imaging system.

In one embodiment, the medical imaging system according to the present embodiments includes a magnetic resonance imaging system, an X-ray imaging system (e.g., a CT-imaging system), an ultrasound imaging system, or any combination thereof.

A magnetic resonance imaging method includes, additionally to the acquisition of raw data, a plurality of processing steps. These processing steps may include processing steps for preparing the raw data for reconstruction of image data, the reconstruction of image data itself, and further steps for generating image data (e.g., the application of additional reconstruction algorithms, the combination of partial images to a combined image, and post-processing steps for corrections of movements). Further, the steps for generating image data may include steps for generating an evaluation of time series, steps for detection of anatomical structures, steps for performing a functional magnetic resonance imaging, steps for performing a diffusion imaging, steps for performing a perfusion imaging, and steps for performing filtering methods. All these steps may be divided in sub-steps (e.g., sub-tasks) and may be performed in a distributed manner using a remote computing source.

In one embodiment, the reconstruction is divided into a plurality of sub-tasks that are transmitted to remote computing resources. Further, also, the following acts like application of additional reconstruction algorithms, post-processing steps for corrections of movements, evaluation of time series, detection of anatomical structures, functional magnetic resonance imaging, diffusion imaging, perfusion imaging, and filtering methods, may be divided into sub-tasks and outsourced to different remote computing sources.

Input data may either be pre-processed raw-data or reconstructed images (e.g., the reconstructed images especially for post-processing), but this is strongly dependent on the concrete algorithm or even variant. Intermediate points may make sense, but in practice, these two are the most relevant ones. For example, both filtering or motion correction may be done either on early stages in the image recon pipeline or on the reconstructed images, depending on the concrete algorithm. In practice, a concrete point is to be identified in the reconstruction pipeline at which it makes sense for the individual concrete use case from which on the external computation would make sense. For example, in case of alternative reconstructions based on the same raw data, this would be at a very early stage of the pipeline (e.g., directly after system-dependent pre-processing/normalization of the raw data), even if the original reconstruction offloaded only, for example, post-processing tasks (e.g., because the medical images, in particular MR images, are acquired sequentially over time).

A CT-imaging method includes the acts of receiving raw data from a scan unit and a preparation step for generating corrected and re-arranged raw data. The CT-imaging method includes an actual CT-reconstruction act for generating image data based on the prepared raw data and a filtering act. In CT-imaging, a plurality of different sets of image data may be generated based on the same set of raw data using different sets of parameters for reconstruction. All these reconstructions may be implemented as sub-tasks performed on different remote computing sources. Further, a filtering act is carried out on the generated image data. Also, the filtering act may be carried out by the different remote computing sources or alternatively by a local control device.

### BRIEF DESCRIPTION OF THE DRAWINGS

The present embodiments are explained below with reference to the figures enclosed once again. The same components are provided with same reference numbers in the various figures.

The figures are usually not to scale.

FIG. 1 shows a magnetic resonance (MR) imaging system according to prior art,

FIG. 2 shows an MR imaging system according to an embodiment;

FIG. 3 shows an ensemble of MR imaging systems according to an embodiment;

FIG. 4 shows an arrangement including an MR imaging system according to an embodiment;

FIG. 5 shows a flow chart diagram illustrating a method for controlling a medical imaging system according to an embodiment;

FIG. 6 shows a schematic view on a local control device according to an embodiment;

FIG. 7 shows acts 5.II to 5.IX illustrated in FIG. 5, schematically represented for an even more detailed understanding;

FIG. 8 illustrates an example of a plan for performing a reconstruction task;

FIG. 9 shows a schematic view on an example of a workflow of the reconstruction;

FIG. 10 shows a roughly schematic representation of one embodiment of a magnetic resonance tomography system;

FIG. 11 shows a flow chart diagram illustrating an embodiment of the method for controlling a medical imaging system applied to a parallel MR-imaging method; and

FIG. 12 shows a flow chart diagram illustrating an embodiment of the method for controlling a medical imaging system applied to a computer tomography method.

### DETAILED DESCRIPTION

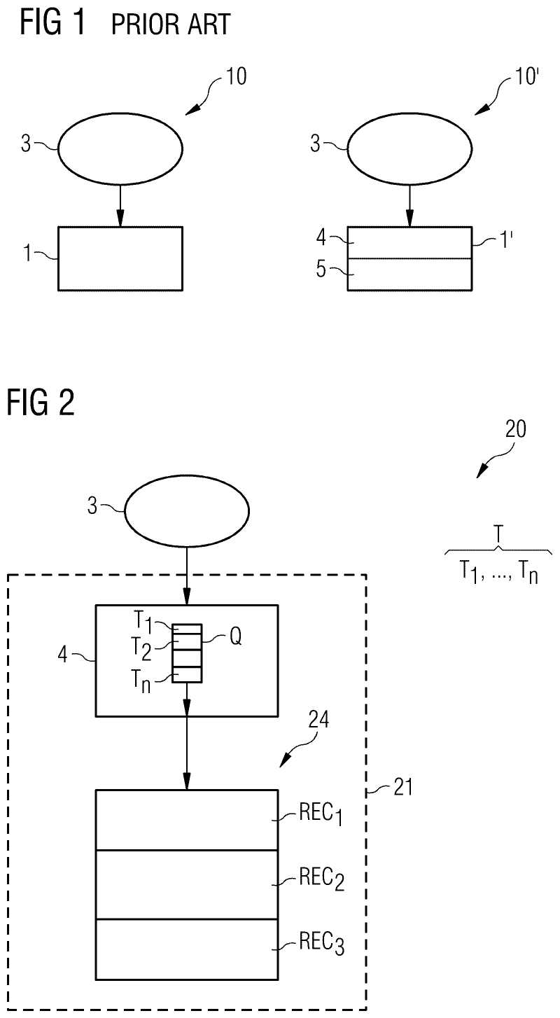

In FIG. 1, a schematic view of two conventional medical imaging systems, a first conventional medical imaging system 10 (e.g., a magnetic resonance (MR) system) and a second conventional medical imaging system 10′ (e.g., an MR system, with a scan unit 3 and an associated control device 1, 1′, respectively, (e.g., measurement and reconstruction system (MARS)) are shown. The first conventional medical imaging system 10 includes a uniform control device 1, and the second conventional medical imaging system 10′ includes a modularized control device including a first unit 4 (e.g., measurement control (MC) unit 4) and a second unit 5 (e.g., reconstruction unit 5 (RECON)). Both types of conventional medical imaging systems 10, 10′ include a monolithic computing model for image reconstruction (e.g., run the whole computation like the image reconstruction as a single complex computing task). The modularized control device 1′ of the second conventional medical imaging system 10′ may be implemented as a logical separation within one single control device or as two real separate entities (e.g., two separate computing units 4, 5). Hence, particularly, the reconstruction tasks in the second conventional medical imaging system 10′ are performed by a monolithic reconstruction unit 5.

FIG. 2 shows a schematic view of a medical imaging system 20 according to an embodiment. The medical imaging system 20 (e.g., a magnetic resonance imaging system) includes a scan unit 3 and a local control device 21. Similar to the second variant 1′ of a conventional control device for a medical imaging system in FIG. 1, the local control device 21 in FIG. 2 includes two separate units 4, 24 (e.g., a measurement control unit 4 and a modularized reconstruction unit 24, also referred to as local control unit). The modularized reconstruction unit 24 includes a plurality of sub-units REC₁, . . . , RECₘ (e.g., computing sources) for performing reconstruction sub-tasks (e.g., RECON-machines) (m is a natural number). For example, the total imaging generation process includes inter alia a reconstruction task T, where, for example, a complete database of input data for that special reconstruction task T is already available. As previously discussed, such a reconstruction task may include the parallel reconstruction of a predetermined number of sub-images based on raw data RD₁, . . . , RDₙ acquired in parallel. This raw data represents the complete data base for the different sub-tasks T₁, . . . , Tₙ of the reconstruction task T.

Some of the different sub-units REC₁, . . . , RECₘ (e.g., with m<=n) perform different computation tasks T₁, . . . , Tₙ. Hence, a reconstruction task T is split into a number of independent computation tasks T₁, . . . , Tₙ, where some of the different independent computation tasks T₁, . . . , Tₙ are performed by a different computing source (e.g., a different sub-unit REC₁, . . . , RECₘ). After computation, the results of the different independent computation sub-tasks T₁, . . . , Tₙ are combined altogether to a combined final overall single result.

The sub-units (e.g., RECON-machines REC₁, . . . , RECₘ) may include individual computers, but the sub-units may also be implemented by a single machine that includes a number of integrated entities or even a single MARS-computer, where each function of the different RECON-machines REC₁, . . . , RECₘ is integrated but still logically separated.

Hence, the principle of the modularized reconstruction unit 24 in FIG. 2 is to split up a monolithic reconstruction task T into certain chunks of independent computation sub-tasks T₁, . . . , Tₙ that may be flexibly either executed on a local machine (e.g., the reconstruction unit 24) or distributed to other places (e.g., remote computing sources or remote medical imaging systems). The whole reconstruction process may include much more sub-tasks than the mentioned sub-tasks T₁, . . . , Tₙ. However, the generated sub-tasks T₁, . . . , Tₙ are already supplied with a necessary data basis of input data such that the generated sub-tasks T₁, . . . , Tₙ may be transferred together with the assigned data basis of input data to a logically separated or physically separated computing source (e.g., a separate RECON-machine REC₁, . . . , RECₘ).

The software structure used for the implementation of the distributed structure is a queue Q, where the reconstruction is divided into the above-mentioned sub-tasks T₁, . . . , Tₙ, also referred to as chunks. These chunks are stacked up and then distributed to different computing sources. The queue Q only includes these selected sub-tasks T₁, . . . , Tₙ ready to be processed at the point of time of selection. Indeed, the individual results of the selected sub-tasks T₁, . . . , Tₙ are collected and put together to the final overall result in a reverse manner. The division of the tasks into sub-tasks T₁, . . . , Tₙ is organized by the measurement control unit 4. The queue Q is symbolized in the measurement control unit 4. The queue Q symbolizes that the different reconstruction sub-tasks T₁, . . . , Tₙ are stacked up and then distributed. The individual results of the different sub-tasks T₁, . . . , Tₙ are then collected and put together to the final overall result in a reverse manner by the local control device 21. The overall result (e.g., of an MR measurement) may be either a simple collection of individual results (e.g., images) for this single MR measurement or something that needs to be (e.g., at least partly) computationally combined or further processed. The first option is typically the most relevant in practice.

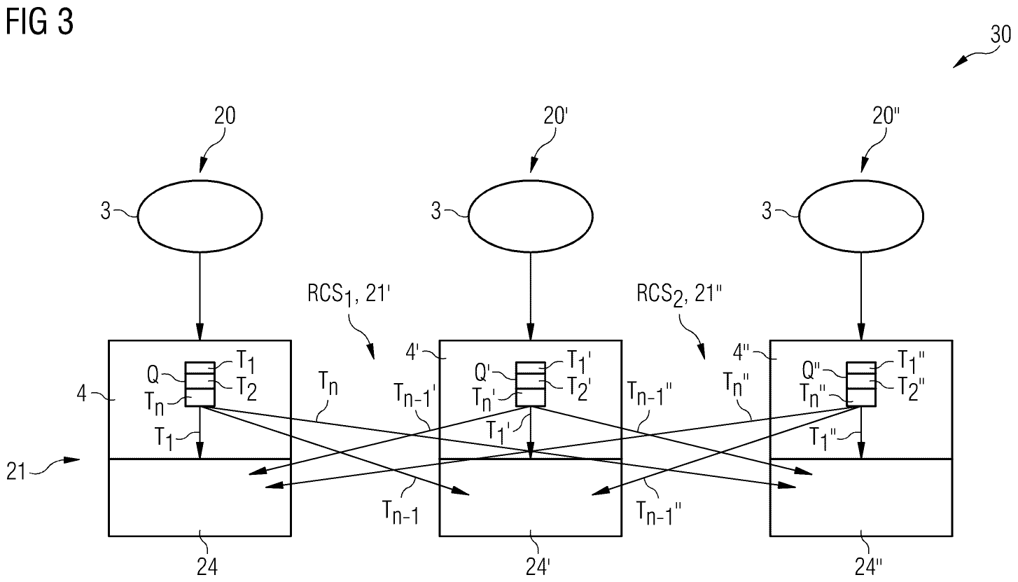

In FIG. 3, an ensemble 30 of a plurality of possibly remotely located medical imaging systems 20 (e.g., three MR imaging systems 20) is visualized. Each of the MR imaging systems 20′, 20″ is structured similar to the MR imaging system 20 shown in FIG. 2. Hence, each of the three MR imaging systems 20, 20′, 20″ includes a control device 21, 21′, 21″, where it depends on the perspective of the control device 21, 21′, 21″ if it is local or remote. Each control device 21, 21′, 21″ includes a measurement control unit 4, 4′, 4″ and a modularized reconstruction unit 24, 24′, 24″. Although not shown, each of the modularized reconstruction units 24, 24′, 24″ are also divided into separate real or logic functional units (e.g., RECON-machines REC₁, . . . , RECₘ) in a cluster-style to process the chunks (e.g., the sub-tasks T₁, . . . , Tₙ of a computing work, such as a reconstruction task T).

However, in FIG. 3, the different reconstruction sub-tasks T₁, . . . , Tₙ are now additionally to the local control unit or reconstruction unit 24, at least partly, distributed to the other free computing resources (e.g., reconstruction units 24′, 24″ of the different MR imaging systems 20′, 20″ used as remote computing source RCS₁, RCS₂). Every MR imaging system 20″ that has an idle computing power may be used to execute such chunks of computing work (e.g., sub-tasks T₁, . . . , Tₙ), and thus, the MR imaging systems 20, 20′, 20″ form a kind of virtual cluster, typically distributed and dynamically available.

Some of these computing sub-tasks T₁, . . . , Tₙ are dynamically transmitted to the other MR imaging systems 20′, 20″ that are available and have free computing resources available. In a worst case, the original MR imaging system 20 performs all of the computing sub-tasks T₁, . . . , Tₙ alone. However, as soon as at least one or more computing sub-tasks T₁, . . . , Tₙ are successfully executed by other supporting MR imaging systems 20′, 20″, the overall image reconstruction time is sped up accordingly. Vice versa, the other MR imaging systems 20′, 20″ also distribute their sub-tasks T′₁, . . . , T′ₙ, T″₁, . . . , T″ₙ each structured in a software structure of a queue Q′, Q″ to remote imaging systems 20, 20″ or remote imaging systems 20, 20′, respectively, for execution.

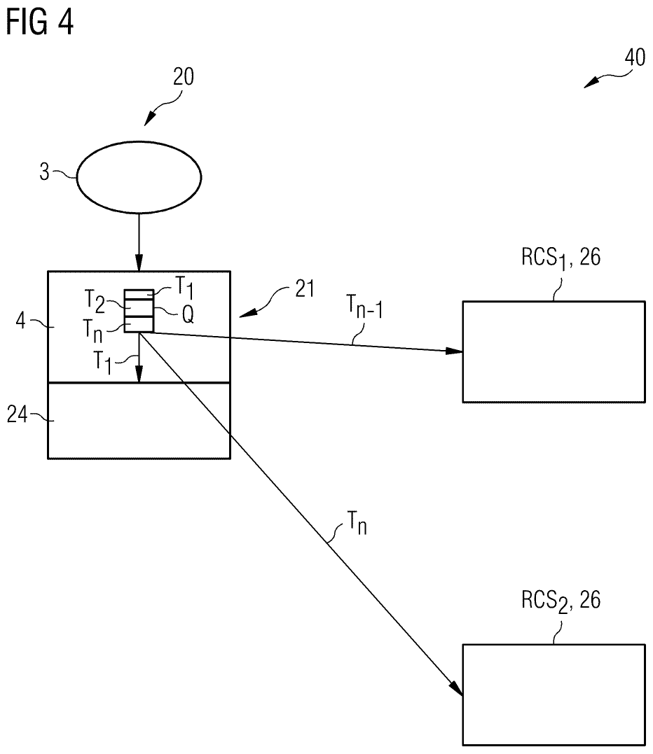

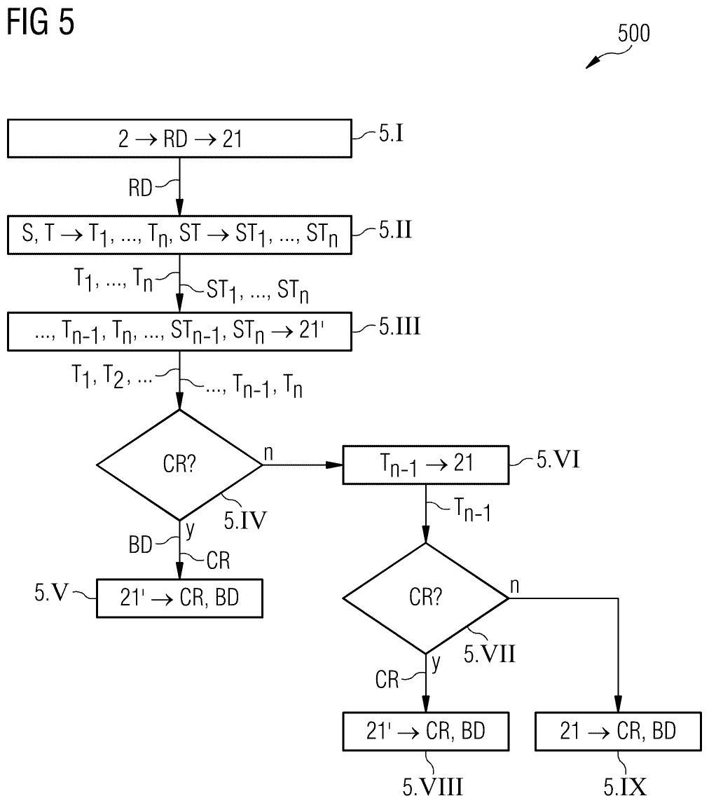

In FIG. 4, a combination 40 (e.g., a dedicated computing cluster of an MR imaging system 20 according to the present embodiments and additional remotely located dedicated compute-only units 26 used as remote computing resources RCS₁, RCS₂) is illustrated. The distribution of computing power amongst each other is enriched by dedicated compute-only units 26 to further speed up computing time. Hence, a dedicated computing cluster includes a plurality of highly-performant computing machines (e.g., compute-only units 26 with a single task of offering free computing resources that may be used by the MR imaging system 20 is realized). As shown in FIG. 4, the different sub-tasks Tₙ₋₁, Tₙ are transmitted to the compute—only units 26, where other sub-tasks T₁, T₂ remain in the MR imaging system 20 such that the MR imaging system 20 may start with these sub-tasks T₁, T₂. In parallel, the other sub-tasks Tₙ₋₁, Tₙ are processed in the compute-only units 26. As soon as these sub-tasks Tₙ₋₁, Tₙ are finished, the remote compute-only units 26 continue with other not yet processed sub-tasks Tₙ₋₂ and so forth, until all tasks are finished.

After finishing the local processed sub-tasks T₁, T₂, the local control device 21 checks if the results of the remotely processed sub-tasks Tₙ₋₁, Tₙ have been finished. Otherwise, the local reconstruction unit 24 of the MR imaging system processes these not yet finished sub-tasks Tₙ₋₁, Tₙ by default, such that the reconstruction process is not interrupted or delayed.

In FIG. 5, a flow chart diagram 500 illustrating one embodiment of a method for controlling a medical imaging system (e.g., a magnetic resonance imaging system 20 (as depicted in FIGS. 2 to 4)) is shown.

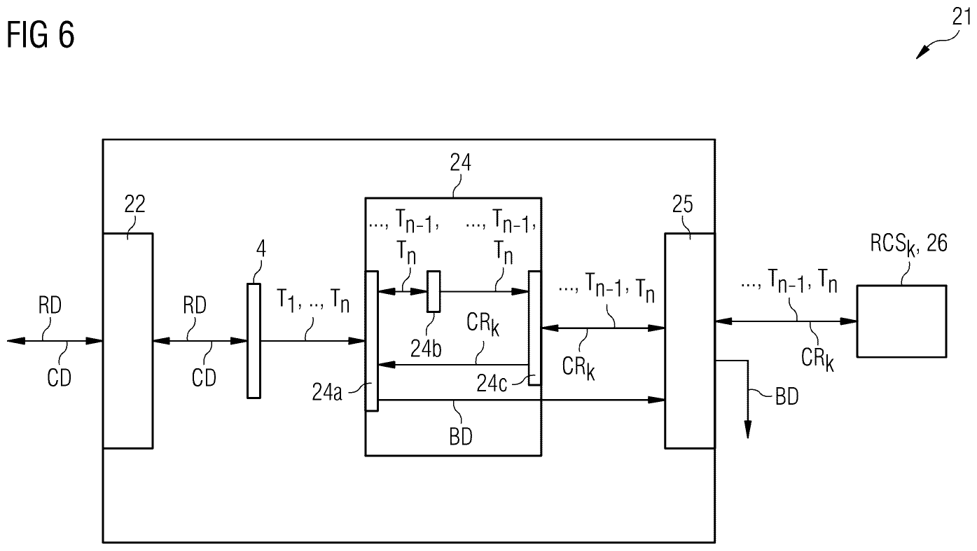

In act 5.I, a scan unit 2 of the magnetic resonance imaging system 20 is controlled by a local control device 21 (shown in FIG. 6) through a control interface 22 (shown in FIG. 6) for generating magnetic resonance signals that are detected as raw data RD and are sent back to the local control device 21 through the control interface 22.

In act 5.II, a computing task T (e.g., a predetermined reconstruction sequence task T for reconstructing images based on raw data acquired in parallel) that includes a plurality of sub-tasks T₁, . . . , Tₙ (e.g., parallel reconstruction steps) is started for reconstructing image data BD. The sub-tasks T₁, . . . , Tₙ are planned to be carried out based on a predetermined sequence S. The sequence S may be determined based on the time the sub-tasks T₁, . . . , Tₙ probably takes. Then, the sub-tasks with less time consumption are assigned to the local computing source, and the more time consuming sub-tasks are assigned to the remote computing sources.

The sub-tasks . . . , Tₙ₋₁, Tₙ later to be carried out in the sequence S are particularly appropriate for sourcing out to a local control device 21′ (shown in FIG. 3) of a remote magnetic resonance imaging system 20′ (shown in FIG. 3). Hence, these sub-tasks . . . , Tₙ₋₁, Tₙ are selected for transmitting these sub-tasks . . . , Tₙ₋₁, Tₙ to the remote magnetic resonance imaging system 20′. In this way, the sub-tasks T₁, T₂, . . . that are on the front positions in the sequence S may be processed in the local control device 21 without any hesitation, and the remote magnetic resonance imaging system 20′ has more time to process the sub-tasks being on the rear positions of the sequence S.

The computing task T is executed based on a set ST of input data each related to a different sub-task T₁, . . . , Tₙ. The set ST of input data, (e.g., raw data) is divided into separate sub-sets ST₁, . . . , STₙ to assign each of the separated sub-sets ST₁, . . . , STₙ to the related sub-task T₁, . . . , Tₙ.

In act 5.III, the selected sub-tasks . . . , Tₙ₋₁, Tₙ are transmitted together with the related sub-sets . . . , STₙ₋₁, STₙ to a remote control device 21′ of the remote magnetic resonance imaging system 20′, and the remaining sub-tasks (e.g., the sub-tasks T₁, T₂, . . . not selected for remote processing and the related sub-sets ST₁, ST₂ . . . ) are locally processed by the local control device 21.

In act 5.IV, the sequence S has been processed up to the selected and outsourced sub-tasks . . . , Tₙ₋₁, Tₙ. At that point of time, it is checked if computing results CR (e.g., image data BD) related to the selected and outsourced sub-tasks . . . , Tₙ₋₁, Tₙ are received from the remote control device 21′ of the remote magnetic resonance imaging system 20′. If the computing results CR (e.g., image data BD) of the selected and outsourced sub-tasks . . . , Tₙ₋₁, Tₙ are received from the remote control device 21′ of the remote magnetic resonance imaging system 20′, which is symbolized with “y” in FIG. 5, then, the computing results CR (e.g., image data BD) of the selected and outsourced sub-tasks . . . , Tₙ₋₁, Tₙ are combined with other computing results CR from the locally processed sub-tasks T₁, T₂, . . . in act 5.V.

If not all of the computing results CR are received from the remote magnetic resonance imaging system 20′, which is symbolized in FIG. 5 with “n”, then the sub-task Tₙ₋₁ assigned to the not received result CR is automatically started on the local control device 21 in act 5.VI.

In act 5.VII, during locally processing the selected sub-task Tₙ₋₁, it is checked if the computing results CR of the selected sub-task Tₙ₋₁ are received before completing the selected sub-task Tₙ₋₁ on the local control device 21. If the computing results CR (e.g., image data BD) are received from the remote control device 21′ before completing the selected sub-task Tₙ₋₁ on the local control device 21, which is symbolized in FIG. 5 with “y”, then, the local procession of the selected sub-task Tₙ₋₁ is stopped, and the result CR assigned to the selected sub-task Tₙ₋₁ is combined with the results CR of the other sub-tasks in act 5.VIII.

If the computing results CR are not received from the remote control device 21′ before completing the selected sub-task Tₙ₋₁ on the local control device 21, which is symbolized in FIG. 5 with “n”, then the sub-task Tₙ₋₁ is finished in the local control device 21 and the assigned computing results CR (e.g., image data BD) are received from the local control device 21 in act 5.IX. Further, the computing results CR (e.g., image data BD) of the different sub-tasks T₁, . . . , Tₙ and resources are combined for generating a final result (e.g., a combined image) of the reconstruction.

In FIG. 6, a schematic view on a local control device 21 for a medical imaging system 20 (shown in FIGS. 3, 4) is illustrated. The local control device 21 includes a first communication interface 22 for transmitting control data CD to a scan unit 3 (shown in FIGS. 3, 4) of the medical imaging system 20 and for receiving raw data RD from the scan unit 3. Further, the local control device 21 includes a local control unit 24 for performing a computing task T related to the control data CD and/or to the received raw data RD. The execution of the computing task T includes a generation of computing results CR related to the control data CD and/or to the received raw data RD. The local control device 21 also includes a second communication interface 25 for communication with a remote computing source 26 (also shown in FIG. 4). The local control device 21 is arranged to execute a computing task T that is divided into a sequence S of consecutive sub-tasks T₁, . . . , Tₙ and to transmit a sub-task . . . , Tₙ₋₁, Tₙ to the remote computing source RCSₖ (e.g., a compute only unit 26; only one single remote computing source RCSₖ is depicted, however, a plurality of remote computing sources may be used for processing the different sub-tasks . . . , Tₙ₋₁, Tₙ in parallel) through the second communication interface 25, to receive computing results CRₖ carried out by occasion in the remote computing source RCSₖ, and to use the received computing results CRₖ for execution of the computing task T.

In detail, the local control device 21 includes a measurement control unit 4 for dividing a computing task T into a sequence S of consecutive sub-tasks T₁, . . . , Tₙ. Further, the local control device 21 includes a local control unit 24.

In detail, the local control unit 24 includes an execution unit 24a for executing the computing task T divided into consecutive sub-tasks T₁, . . . , Tₙ. The local control unit 24 (e.g., in that embodiment, a reconstruction unit 24), also includes a selection unit 24b for selecting a sub-task . . . , Tₙ₋₁, Tₙ of the consecutive sub-tasks T₁, . . . , Tₙ for external execution and an outsourcing unit 24c for outsourcing the selected sub-task . . . , Tₙ₋₁, Tₙ to a remote computing source RCSₖ (e.g., a compute-only unit 26) for remotely generating a partial computing result CRₖ and for receiving the generated partial computing result CRₖ. The execution unit 24a is arranged to execute the computing task T based on the received generated partial computing result CRₖ and for generating and emitting the final result (e.g., the final image data BD) through the second communication interface 25.

Selecting and outsourcing of the sub-tasks may in principle be done either in the control unit 4 or the reconstruction unit 24.

For the first variant, language that the control unit 4 is close to “systems knowledge” and one can better control the overall behavior, especially since the process is in or at least very close to the real-time activities of the machine (e.g., the scan unit 3). Additionally, the reconstruction unit 24 does not need to be aware of this task of outsourcing and thus may simply concentrate on the task of computing, and thus, all computing units (e.g., including the remote computing source 26) may then be implemented very similar (e.g., this makes the scaling over different hardware much easier).

But also, the other approach, to implement the scheduling of sub-tasks in the reconstruction unit 24, may have advantages: The control-unit 4 is not affected by its main task (e.g., including real-time tasks related to raw data acquisition and pre-processing, which barely make sense to outsource). The reconstruction unit 24 then automatically gets the more complex or comprehensive computing tasks related to image reconstruction only and may (e.g., even better, since reconstruction is its core knowledge) decide on its own, how to split up and distribute computation.

The components of the local control device 21 may be implemented predominantly or completely in the form of software elements on a suitable processor. For example, the interfaces between these components may also be configured purely in terms of software. That is provided if there are access options to suitable storage areas in which the data may be stored temporarily and called up and updated at any time.

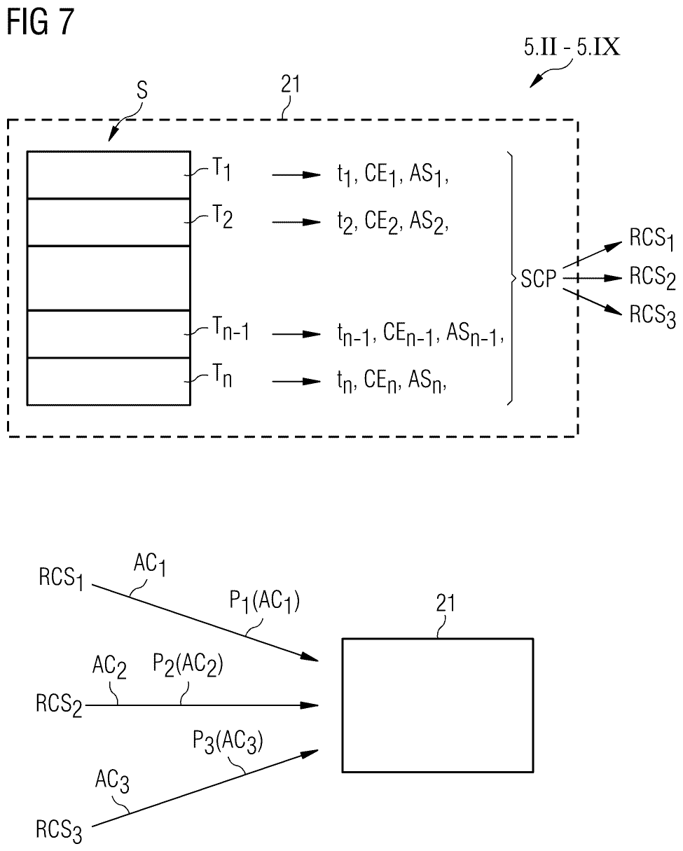

In FIG. 7, acts 5.II to 5.IX illustrated in FIG. 5 are schematically represented for an even more detailed understanding of the principles of the execution of the method for controlling a medical imaging system according to the present embodiments.

In act 5.II, a computing task T related to a predetermined reconstruction sequence S that includes a plurality of sub-tasks T₁, . . . , Tₙ (e.g., reconstruction steps) is planned. The sub-tasks T₁, . . . , Tₙ are planned to be carried out based on the predetermined sequence S. For planning the computing task T, a set SCP of characteristic parameters for each subtask T₁, . . . , Tₙ is calculated. To each of these individual sub-tasks T₁, . . . , Tₙ, a time interval t₁, . . . , tₙ for the time requirement for performing each individual sub-task T₁, . . . , Tₙ is assigned, and an estimated computational effort CE₁, . . . , CEₙ and amount of storage AS₁, . . . , ASₙ is calculated based on the type of the individual sub-task T₁, . . . , Tₙ and the received amount of raw data RD. The computational effort CE₁, . . . , CEₙ may be calculated based on the complexity of an algorithm related to the respective sub-task T₁, . . . , Tₙ and the amount of raw data RD. Hence, the set SCP of characteristic parameters includes the following parameters:
time interval t: t₁, . . . , tₙ,
computational effort CE: CE₁, . . . , CEₙ,
amount of storage AS: AS₁, . . . , ASₙ.

The calculation of the set SCP of characteristic parameters may also be performed based on experimental or empiric data.

Further, a request is started and transmitted to all potential remote computing sources RCSₖ (k=1 . . . , m, in FIG. 7: m=3) (shown in FIG. 3 and FIG. 4), where the determined set SCP of characteristic parameters is transmitted to each of the remote computing sources RCSₖ.

After that, each of the requested remote computing sources RCSₖ replies by transmitting an answer including the free capacities for performing at least one or more sub-tasks T₁, . . . , Tₙ.

Optionally, each remote computing source RCSₖ calculates itself based on the set SCP of characteristic parameters, which of the sub-tasks T₁, . . . , Tₙ the respective remote computing source RCSₖ is able to perform in which amount of time (e.g., in which time interval t₁ₖ, tₙₖ). Alternatively, each remote calculating source RCSₖ transmits a set representing its available capacity ACₖ, (e.g., including its computation performance CPₖ, its available amount of storage ASₖ, and its available calculation time atₖ) and, for example, the price Pₖ(ACₖ) for the usage of the available capacity.

Based on the set SCP of characteristic parameters, a set of the received information related to available capacities ACₖ and a set of received assigned prices Pₖ(ACₖ), an optimization, for example, by generating a time function t_{F}=f(T, SCP, ACₖ) and a cost function c_{F}=f(Pₖ(ACₖ)) may be determined in the local control device 21.

Further, it has to be respected that an individual sub-task T₁, . . . , Tₙ may be only started if the directly preceding sub-task T₁, . . . , Tₙ has been finished, if there are data dependencies.

Taking into account that information, an optimal plan for performing the reconstruction task T is completed.

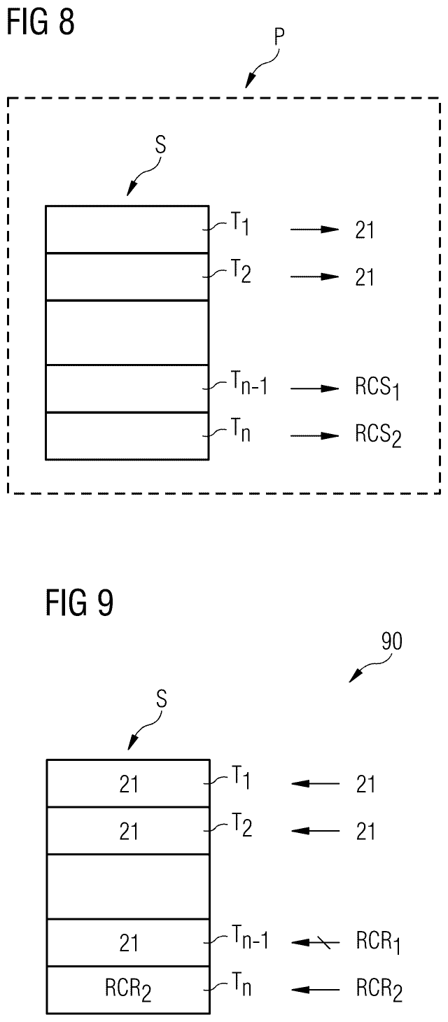

Such a plan P is shown in FIG. 8. To each of the sub-tasks T₁, . . . , Tₙ an individual calculating source is assigned. For example, the first sub-task T₁, and the second sub-task T₂ are performed by the local control device 21. The n−1-th sub-task Tₙ₋₁ is performed by a first remote calculating source RCS₁, and the n-th sub-task Tₙ is performed by a second remote calculating source RCS₂.

After that, a reconstruction T is started. In FIG. 9, a schematic view 90 on the workflow of the reconstruction task T is illustrated. After finishing the n−2-th sub-task, the first remote calculating resource RCS₁ has not finished its calculation (e.g., due to a problem of transmitting data). Hence, the local control device 21 starts itself vicariously performing the n−1-th sub-task Tₙ₋₁ and generates results CRₙ₋₁ or distributes it to another remote computing device if, for example, that one is much more powerful than it is itself. At last, the n-th sub-task Tₙ is finished just in time by the second remote calculating source RCS₂, and the reconstruction task T may be completed based on the result of the n-th sub-task Tₙ finished by the second remote computing source RCS₂, and all computing results are combined. Consequently, the reconstruction task T is finished in reality in an optimum of time and optionally for an optimized amount of costs.

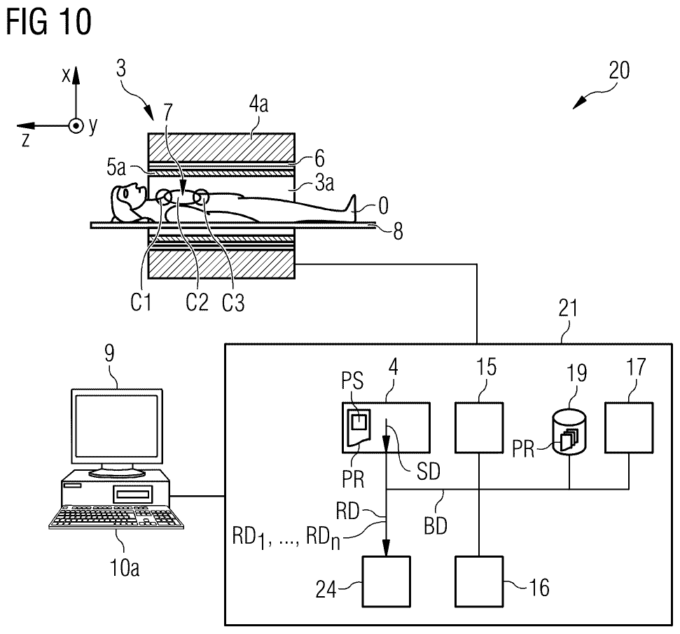

FIG. 10 shows a roughly schematic representation of a magnetic resonance imaging system 20 according to the present embodiments (hereinafter also referred to as “MR system” for short), including a local control device 21 that is configured to carry out a method for controlling an MR system according to the present embodiments. The MR system includes the actual magnetic resonance scan unit 3 with an examination room 3a or patient tunnel, in which an examination object O, or, for example, a patient or test person, in whose body the examination object O (e.g., a specific organ located) may be retracted, is placed on a bed 8.

The magnetic resonance scan unit 3 is equipped in the usual way with a basic field magnet system 4a, a gradient system 6, and an HF transmission antenna system 5a and an HF reception antenna system 7. In the exemplary embodiment shown, the HF transmission antenna system 5a is a whole-body coil permanently installed in the magnetic resonance scan unit 3. The HF reception antenna system 7 includes local coils with reception coils C1, C2, C3 to be arranged on the patient or subject O (e.g., in FIG. 10, this is symbolized by three receiving coils C1, C2, C3, typically, there are a number of receiving coils). The receiving coils C1, C2, C3 may be grouped in units with a common housing, which are often referred to as local coils. A number of reception coils are available for parallel measurement.

The basic field magnet system 4a is configured, for example, in the usual way so that there is a basic magnetic field in the longitudinal direction of the examination object O (e.g., a patient; along the longitudinal axis of the magnetic resonance scan unit 3 running in the z-direction). The gradient system 6 includes gradient coils that may be controlled individually in the usual way, in order to be able to switch gradients in the x, y, or z direction independently of one another.

The MR system 20 also has a local control device 21 that is used to control the MR system 20. This central local control device 21 includes a sequence control unit or measurement control unit 4 for measurement sequence control. This is used to record the sequence of high-frequency pulses (HF pulses) and gradient pulses as a function of a selected pulse sequence PS or a sequence of multiple pulse sequences for recording multiple slices or volumes in a volume region of interest of the examination object O.

Such a pulse sequence PS may be specified and parameterized within a measurement or control protocol PR, for example. Different control protocols PR for different measurements or measurement sessions are usually stored in a memory 19 and may be selected by an operator (and changed if necessary) and then used to carry out the measurement. In the present case, a pulse sequence is selected such that raw data is acquired in parallel with a plurality of receiving coils in the desired raster in a subsampled manner, and for each coil, a different raw data set RD₁, . . . , RDₙ is received.

To output the individual HF pulses of a pulse sequence PS, the local control device 21 has a high-frequency transmission device 15 that generates and amplifies the HF pulses and feeds the HF pulses into the HF transmission antenna system 5a via a suitable interface (not shown in detail). The local control device 21 has a gradient system interface 16 for controlling the gradient coils of the gradient system 6 in order to switch the gradient pulses appropriately according to the predetermined pulse sequence. The measurement control unit 4 communicates in a suitable manner (e.g., by sending sequence control data SD) with the high-frequency transmission device 15 and the gradient system interface 16 for executing the pulse sequences. The local control device 21 has also a high-frequency receiving device 17 (e.g., that also communicates in a suitable manner with the measurement control unit 4). The receiving device 17 receives magnetic resonance signals within the readout window specified by the pulse sequence PS in a coordinated manner using the HF reception antenna system 7 to acquire the raw data RD, including raw data sets RD₁, . . . , RDₙ after demodulation and digitization.

A reconstruction unit 24 accepts the acquired raw data sets RD₁, . . . , RDₙ at a raw data interface (e.g., a receiving unit), and reconstructs image data BD for the desired visual field from the acquired raw data sets RD₁, . . . , RDₙ. This reconstruction may also be based on parameters that are specified in the respective measurement protocol.

In the present case, the reconstruction unit 24 is configured such that the reconstruction unit 24 may work according to the method according to present embodiments (e.g., for parallel reconstruction based on the different receiving coils C1, C2, C3).

The local control device 21 may be operated via a terminal with an input unit 10a and a display unit 9, via which the entire MR system 20 may therefore also be operated by an operator. MR images may also be displayed on the display unit 9, and measurements may be planned and started using the input unit 10a, possibly in combination with the display unit 9, and, for example, control protocols PR with suitable pulse sequences PS may be selected as explained above and modified if necessary.

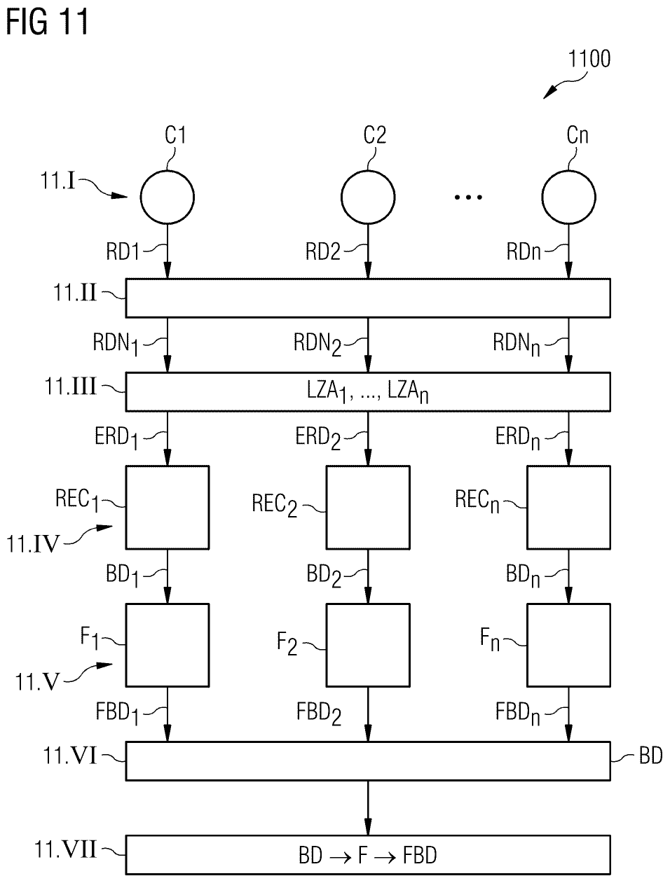

In FIG. 11, a flow chart diagram 1100 illustrating an embodiment of the method according to the present embodiments related to parallel MR imaging is shown.

First, in act 11.I, raw data sets RD₁, . . . , RDₙ are acquired on different channels using a plurality of reception coils C1, C2, . . . , Cn arranged on a patient. In act 11.II, the different raw data RD₁, . . . , RDₙ are then normalized in the local control device 21.

After that, the normalized raw data RDN₁, . . . , RDNₙ still includes blank lines or empty lines. Therefore, in act 11.III, sub-tasks LZA₁, . . . , LZAₙ for adding empty lines are applied on the normalized raw data RDN₁, . . . , RDNₙ such that complemented raw data ERD₁, . . . , ERDₙ are generated. Also, these sub-tasks LZA₁, . . . , LZAₙ are usually locally performed due to their relatively low calculating amount.

Then, in act 11.IV, the complemented raw data ERD₁, . . . , ERDₙ is assigned to different reconstruction sub-tasks REC₁, . . . , RECₙ, and some of these sub-tasks REC₁, . . . , RECₙ are then transmitted to remote computing sources. Further, image data BD₁, . . . , BDₙ is generated based on the complemented raw data ERD₁, . . . , ERDₙ by the local control device 21 and also by the remote computing sources.

Further, in act 11.V, the generated image data BD₁, . . . , BDₙ results are assigned to filtering sub-tasks F₁, . . . , Fₙ, and the filtering sub-tasks F₁, . . . , Fₙ and the generated partial image data BD₁, . . . , BDₙ are transmitted to remote computing resources for generating filtered image data FBD₁, . . . , FBDₙ. After that, in act 11.VI, the filtered image data FBD₁, . . . , FBDₙ is combined to a combined image BD by the local control device 21. In act 11.VII, an additional filter step F is applied to the combined image BD by the local control device 21.

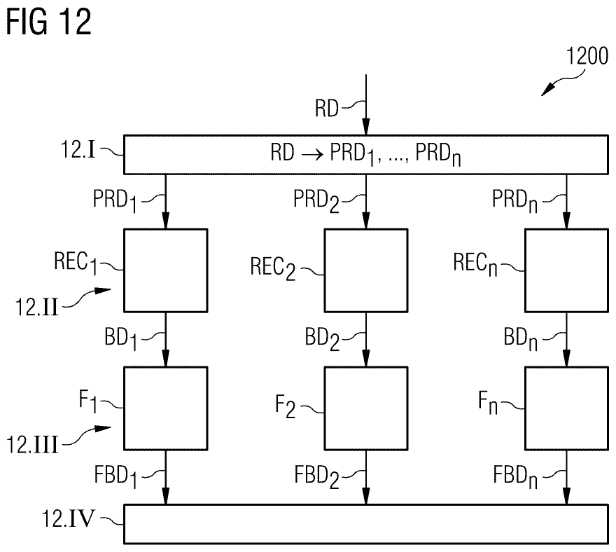

In FIG. 12, a flow chart diagram 1200 illustrating an embodiment of the method according to the present embodiments related to CT imaging is shown.

In act 12.I, raw data RD is received by a local control device from a scan unit of a CT-imaging system. Further, the raw data RD is locally prepared by correcting and re-arranging the raw data RD such that prepared raw data PRD₁, . . . , PRDₙ is generated.

In act 12.II, a reconstruction task is divided into a plurality of sub-tasks REC₁, . . . , RECₙ assigned to different sets of prepared raw data PRD₁, . . . , PRDₙ and these sub-tasks REC₁, . . . , RECₙ are transmitted to remote computing sources. Further, the prepared raw data PRD₁, . . . , PRDₙ is used for generating reconstructed image data BD₁, . . . , BDₙ.

In act 12.III, the generated image data BD₁, . . . , BDₙ is processed by remotely implemented filters F₁, . . . , Fₙ. The generated filtered image data FBD₁, . . . , FBDₙ is then sent back to the local control device of the CT imaging system.

The above descriptions are merely embodiments of the present disclosure, but not intended to limit the present disclosure, and any modifications, equivalent replacements, improvements, etc. made within the spirit and principle of the present disclosure is to be included within the scope of protection of the present disclosure.

Further, the use of the undefined article “a” or “one” does not exclude that the referred features may also be present a number of times. Likewise, the term “unit” or “device” does not exclude that the unit or device consists of a number of components that may also be spatially distributed.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A local control device for a medical imaging system, the local control device comprising:
a measurement control unit configured to divide a computing task into a sequence of consecutive sub-tasks, wherein the computing task is related to a generation of image data based on measurement data;
an execution unit configured to execute the computing task;
a selection unit configured to select a sub-task of the consecutive sub-tasks for external execution; and
an outsourcing unit configured to:
outsource the selected sub-task to a remote computing source for remotely generating a partial computing result of the selected sub-task; and
receive the generated partial computing result,
wherein the execution unit is configured to execute the computing task based on the received generated partial computing result.

2. The local control device of claim 1, wherein the computing task includes at least one control task, the at least one control task comprising:
control of a scan unit of the medical imaging system;
reconstruction of image data based on raw data acquired by a scan unit of the medical imaging system; or
the control and the reconstruction.

3. The local control device of claim 1, wherein the computing task is executed based on a set of input data, and
wherein the measurement control unit is configured to:
divide the set of input data into sub-sets; and
assign each of the sub-sets to a different sub-task of the consecutive sub-tasks.

4. The local control device of claim 3, wherein the computing task comprises a reconstruction task for parallel image reconstruction based on sub-sets of parallel acquired raw data, and each sub-task of the consecutive sub-tasks comprises a reconstruction of a partial image based on a different sub-set.

5. The local control device of claim 1, wherein the execution unit is further configured to locally execute a sub-task of the consecutive sub-tasks when an outsourcing of the sub-task is not appropriate according to a predefined criterion.

6. The local control device of claim 1, wherein the execution unit is configured to:
detect a status of a completed consecutive sub-task;
determine, based on the detected status, when the partial computing result generated by the remote computing source is received by the local control device or will be received on time; and
execute the computing task based on the received partial computing result when the partial computing result is received on time, or execute the selected sub-task when the partial computing result is not received on time.

7. The local control device of claim 1, wherein the remote computing source includes a remote local control device of a remote medical imaging system.

8. The local control device of claim 1, wherein the remote computing source includes a cluster comprising a plurality of remote local control devices for remote medical imaging systems.

9. The local control device of claim 1, wherein the remote computing source is located in a cloud computing system.

10. The local control device of claim 2, wherein the computing task is executed based on a set of input data, and
wherein the measurement control unit is configured to:
divide the set of input data into sub-sets; and
assign each of the sub-sets to a different sub-task of the consecutive sub-tasks.

11. The local control device of claim 10, wherein the computing task comprises a reconstruction task for parallel image reconstruction based on sub-sets of parallel acquired raw data, and each sub-task of the consecutive sub-tasks comprises a reconstruction of a partial image based on a different sub-set.

12. The local control device of claim 11, wherein the execution unit is further configured to locally execute a sub-task of the consecutive sub-tasks when an outsourcing of the sub-task is not appropriate according to a predefined criterion.

13. A medical imaging system comprising:
a scan unit configured to acquire raw data from an examination object; and
a local control device comprising:
a measurement control unit configured to divide a computing task into a sequence of consecutive sub-tasks, wherein the computing task is related to a generation of image data based on measurement data;
an execution unit configured to execute the computing task;
a selection unit configured to select a sub-task of the consecutive sub-tasks for external execution;
an outsourcing unit configured to:
outsource the selected sub-task to a remote computing source for remotely generating a partial computing result of the selected sub-task; and
receive the generated partial computing result,
wherein the execution unit is configured to execute the computing task based on the received generated partial computing result.

14. A system-cluster comprising:
at least one medical imaging system, a medical imaging system of the at least one medical imaging system comprising:
a scan unit configured to acquire raw data from an examination object; and
a local control device comprising:
a measurement control unit configured to divide a computing task into a sequence of consecutive sub-tasks, wherein the computing task is related to a generation of image data based on measurement data;
an execution unit configured to execute the computing task;
a selection unit configured to select a sub-task of the consecutive sub-tasks for external execution;
an outsourcing unit configured to:
outsource the selected sub-task to a remote computing source for remotely generating a partial computing result of the selected sub-task; and
receive the generated partial computing result,
wherein the execution unit is configured to execute the computing task based on the received generated partial computing result
at least one additional remote system including the remote computing source for remotely generating a partial computing result of the selected sub-task.

15. The system-cluster of claim 14, wherein the additional remote system comprises at least one type of remote system, the at least one type of remote system comprising a medical imaging system, a compute-only source, or the medical imaging system and the compute-only source.

16. A method for controlling a medical imaging system, the method comprising:
dividing a computing task into a sequence of consecutive sub-tasks, wherein the computing task is related to a generation of image data;
executing the computing task;
selecting a sub-task of the consecutive sub-tasks for external execution;
outsourcing the selected sub-task to a remote computing source for remotely generating a partial computing result; and
receiving the generated partial computing result,
wherein executing the computing task comprises executing the computing task based on the received generated partial computing result.

17. In a non-transitory computer-readable storage medium that stores program sections having instructions executable by a computer unit to control a medical imaging system, the instructions comprising:
dividing a computing task into a sequence of consecutive sub-tasks, wherein the computing task is related to a generation of image data;
executing the computing task;
selecting a sub-task of the consecutive sub-tasks for external execution;
outsourcing the selected sub-task to a remote computing source for remotely generating a partial computing result; and
receiving the generated partial computing result,
wherein executing the computing task comprises executing the computing task based on the received generated partial computing result.
